(19) United States
(12) Patent Application Publication
(10) Pub. No.: US 2006/0025633 A1
(43) Pub. Date: Feb. 2, 2006

(54) Catalytic method of producing mercaptans from thioethers

(76) Inventors: Georges Fremy, Sauveterre de Bearn (FR); Nadine Essayem, Saint Just Chaleyssin (FR); Michel Lacroux, Lyon (FR); Elodie Zausa, Targon (FR)

Correspondence Address: ARKEMA INC.;PATENT DEPARTMENT - 26TH FLOOR, 2000 MARKET STREET, PHILADELPHIA, PA, 19103-3222, US

(21) Appl. No.: 10/528,861

(22) PCT Filed: Sep. 23, 2003
(86) PCT No.: PCT/FR03/02790

§ 371 Date: Mar. 23, 2005

(30) Foreign Application Priority Data
Sep. 25, 2002 (FR) 02/11922

(51) Int. Cl. C07C 319/06

(52) U.S. Cl. 568/70

(57) ABSTRACT

The process for preparing a mercaptan from a thioether and hydrogen sulphide is carried out in the presence of hydrogen and a catalyst composition comprising a strong acid, such as a heteropolyacid, and at least one metal belonging to group VIII of the Periodic Table.

The present invention pertains to the field of mercaptans (also called thiols) and relates more particularly to a catalytic process for preparing mercaptans from thioethers and hydrogen sulphide in the presence of hydrogen and a specific catalyst.

The industrial significance of mercaptans or thiols means that many studies have been carried out for the purpose of perfecting the preparation of these compounds. In particular a process is known which is widely employed and which implements the reaction of hydrogen sulphide with an alcohol or an olefin. In such a reaction a by-product which is obtained in particular comprises one or more thioethers, which result from secondary reactions and, primarily, from the reaction of the mercaptan (formed in the main reaction) with the starting reactant, in other words either the alcohol or the olefin, depending on the process used.

Thioethers obtained as by-products during the preparation of mercaptans are not generally of commercial significance.

Methods of converting these thioethers for the purpose of upgrading them have been proposed, the aim of these methods being to transform the thioethers into mercaptans by reaction with hydrogen sulphide (H₂S) in the presence of various catalysts, in a reaction known as sulfhydrolysis.

Existing sulfhydrolysis processes employ this reaction under pressure, using a reaction stream composed exclusively of H₂S and thioether in various proportions, in the presence of various catalyst systems.

Thus U.S. Pat. No. 4,005,149 describes the preparation of mercaptans (or thiols) by reacting H₂S with organic sulphides (another name for thioethers) in the presence, as catalyst, of a sulphide of a metal from group VI and/or of a metal from group VIII, particularly a sulphide of cobalt and molybdenum (Co/Mo) impregnated on an alumina support. Carbon disulphide, CS₂, is added to the reaction mixture in order to improve the conversion of the organic sulphide to mercaptan.

U.S. Pat. No. 4,396,778 describes a vapour-phase process for preparing high molecular weight C₁-C₁₈ alkyl mercaptans using as catalyst a large-pore zeolite modified with potassium or sodium. The reaction is carried out at a high temperature, greater than 290° C.

U.S. Pat. Nos. 2,829,171 and 3,081,353 describe the synthesis of lighter mercaptans such as methyl mercaptan in the presence of activated alumina as catalyst. The reaction temperatures employed in these processes are high.

Highly acidic ion exchange resins as described in U.S. Pat. No. 4,927,972 are catalysts which are also employed in thioether sulfhydrolysis processes, but they generally lead to a low yield.

U.S. Pat. No. 4,059,636 describes the use of a solid catalyst comprising a 12-phosphotungstic acid supported on alumina. This catalyst, compared with a customary catalyst such as molybdenum and cobalt supported on alumina (CoMo/Al₂O₃), has the effect of higher conversion and higher selectivity when it is employed in the sulfhydrolysis reaction, and achieves this with a lower reaction temperature. It may, however, require the presence of carbon sulphide, CS₂, as promoter. No indication is given regarding the stability over time of this catalyst system.

A solid catalyst comprising a 12-phosphotungstic acid supported on silica is also described by U.S. Pat. No. 5,420,092. That document teaches, more generally, the use of a heteropolyacid in combination with a metal from group VIII, but in the distant field of the isomerization of paraffins.

A new catalytic process has now been found for preparing a mercaptans from thioethers and hydrogen sulphide, which employs hydrogen in the reaction stream and a specific catalyst. It has the advantage of utilizing lower temperatures, of obtaining high-purity mercaptans with a good yield, and of maintaining the high activity of the catalyst over time.

The invention accordingly provides a process for preparing a mercaptan from a thioether and hydrogen sulphide, characterized in that it is carried out in the presence of hydrogen and a catalyst composition comprising a strong acid and at least one metal belonging to group VIII of the Periodic Table.

The combination of the hydrogen with this catalyst composition allows the activity of the catalyst to be stabilized at a high level over time and at a relatively low temperature. This result is all the more surprising for being obtained in a sulphurizing medium, which is known to poison the active sites of catalysts.

The strong acid which can be used in the catalyst composition is selected from the group consisting of:
(a) one or more heteropolyacids selected from:
   (i) a compound of formula: H₃PW₁₂O₄₀.nH₂O, H₄SiW₁₂O₄₀.nH₂O or H₆P₂W₁₈O₆₂.nH₂O, in which n is an integer representing the number of molecules of water of crystallization, and (for a commercial product) is generally between 0 and 30, preferably between 6 and 20;
   (ii) a potassium, rubidium, caesium or ammonium salt of at least one compound (i), or a mixture of such salts;
(b) a sulphated zirconium oxide,
(c) a tungstic zirconium oxide,
(d) a zeolite, and
(e) a cationic resin.

The heteropolyacid (i) is generally obtained by condensing two or more different oxo acids, such as phosphoric acid, silicic acid or tungstic acid. It is soluble in water or in a polar organic solvent. The compound of formula H₃PW₁₂O₄₀.nH₂O is known under the name of 12-phosphotungstic or 12-tungstophosphoric acid and is available commercially. The compound of formula H₄SiW₁₂O₄₀.nH₂O is known under the name of 12-tungstosilicic or 12-silicotungstic acid, and is likewise available commercially. The compound of formula H₆P₂W₁₈O₆₂.nH₂O can be prepared according to the procedure described in the following reference: A. P. Ginsberg, Inorganic Synthesis, Vol. 27, published by J. Wiley & Sons (1990) pages 105-107.

The heteropolyacid (ii) is a salt obtained by partial substitution of one or more protons of the heteropolyacid (i) by the corresponding cation. It is evident to the skilled person that such substitution cannot be total without the acidity being lost. A salt of this kind is prepared from a solution of the heteropolyacid (i), to which the desired amount of the alkali metal or ammonium precursor is added. The preferred precursor is the corresponding chloride or carbonate. The precipitated salt is separated off and then dried under gentle conditions, preferably by centrifugation followed by lyophilization. One reference which may be mentioned is the following: N. Essayem, G. Coudurier, M. Fournier, J. C. Vedrine, Catal. Lett., 34 (1995) pages 224-225.

The sulphated zirconium oxide (b) is prepared by impregnating sulphuric acid on a zirconium oxide support in accordance with the process described in the following reference:

F. R. Chen, G. Coudurier, J-F Joly and J. C. Vedrine, J. Catal., 143 (1993) page 617.

The tungstic zirconium oxide (c) is prepared by impregnating tungsten oxide on a zirconium oxide support, in accordance with the process described in U.S. Pat. No. 5,113,034 to Soled et al.

According to a first embodiment of the process according to the invention the catalyst employed in the said process comprises as strong acid a heteropolyacid (ii), or one of the compounds (b), (c), (d) or (e). This version is preferred because, owing to the specific surface properties of a strong acid of this kind, it is generally suitable as a support. It is therefore not necessary in this case to deposit the strong acid on a support.

The catalyst composition comprises in this case:

from 90% to 99.9%, preferably from 98.5% to 99.5%, by weight of strong acid, and

from 0.01% to 10%, preferably from 0.05% to 1.5%, by weight of metal from group VIII.

According to a second embodiment the catalyst employed comprises as strong acid a heteropolyacid (i). This version is preferred owing to the particularly advantageous activity of the catalyst in the sulfhydrolysis reaction.

The catalyst composition comprises in this case:

from 10% to 60%, preferably from 25 to 50%, by weight of strong acid,

from 0.01% to 10%, preferably from 0.1% to 2%, by weight of metal from group VIII, and

from 30% to 80%, preferably from 48% to 75%, by weight of a support selected from silica SiO₂, alumina Al₂O₃, titanium dioxide TiO₂, zirconium oxide ZrO₂, and activated carbon.

According to one particularly preferred embodiment the strong acid employed in the catalyst is 12-phosphotungstic acid, preferably impregnated on silica.

The metal or metals belonging to group VIII of the Periodic Table that is or are generally included in the catalyst composition employed is or are selected from, in particular, iron, cobalt, nickel, ruthenium, rhodium, palladium, osmium, iridium, and platinum.

Preference is given to employing a metal from group VIII that is selected from palladium, ruthenium and platinum, and is especially palladium.

One particularly preferred catalyst composition is that comprising approximately 40% by weight of 12-phosphotungstic acid, 1% of palladium and 59% of silica.

The catalyst composition employed in a process according to the invention may be prepared generally as follows:

When the strong acid used is one of the compounds (i):

(1) the support is heat-treated under vacuum at a temperature of between 90 and 150° C., preferably of around 100° C., and then

(2) the support thus treated is impregnated with an aqueous or organic solution of acid pH, containing the compound (i) and an acidic precursor of the metal from group VIII, and then

(3) the solid thus obtained is dried, and then

(4) is treated with H₂ at a temperature of between 80 and 300° C., preferably between 180 and 250° C.

The aim of the heat treatment of step (1) is to desorb the water which may have been adsorbed in the pores of the support.

In step (2) the acidic precursor refers to a compound which in aqueous solution gives rise to a cationic or anionic complex of the said metal. Examples of such compounds, in the case of platinum, are as follows: tetraammineplatinum hydroxide, tetraammine platinum chloride, dinitrodiamine-platinum(II), or else, in the case of palladium: palladium chloride, Pd(NH₃)₄Cl₂, (NH₄)₂(PdCl₄). Examples of such compounds further include, in the case of platinum: hexachloroplatinic acid (also called hydrogen hexachloroplatinate(IV)), ammonium tetrachloroplatinate(II), and ammonium hexachloroplatinate(IV). The list of acidic precursors is given above purely by way of illustration, without limiting the compounds which can be used as an acidic precursor by the skilled person.

In step (3) the drying may be carried out, for example, by heating the impregnated support, where appropriate under vacuum, at a temperature of generally between ambient temperature and 120° C. for a time ranging from 30 minutes to 5 hours.

The H₂ treatment of step (4) is advantageously carried out on the catalyst when the latter has been placed in the sulfhydrolysis reactor, and its purpose is to reduce the acidic precursor to metal from group VIII.

When the catalyst employed comprises as strong acid a heteropolyacid (ii), or one of the compounds (b), (c), (d) or (e), it may be prepared by the same process except for the fact that the heat treatment is not mandatory, and must even be suppressed or modified, depending on the characteristics of the support.

The catalyst composition described above is employed in the process for preparing mercaptan according to the invention, which comprises reacting hydrogen sulphide (H₂S) with a thioether in the presence of hydrogen.

This process is carried out in the gas phase, insofar as the temperature and pressure conditions utilized are such that the reactants and the products are in the gaseous state.

The hydrogen is introduced into the process in an amount corresponding to a molar H₂S/H₂ ratio of between 10 and 200, preferably between 50 and 100.

The thioether (or organic sulphide) used as starting reactant has the general formula:
R—S—R′ (I)

in which R and R′, which are identical or different, represent an alkyl radical of 1 to 20 carbon atoms, preferably 1 to 12 carbon atoms, which is linear or branched, or else a cycloalkyl radical of 3 to 7 carbon atoms.

Preference is given to using as starting thioether a compound of formula (I) in which R and R′ are identical. This is because, in this case, there is no need to separate the thiols obtained.

The thioether more preferably used is diethyl sulphide (or ethyl thioether). The sulfhydrolysis reaction leads in this case to ethyl mercaptan (or ethanethiol).

The hydrogen sulphide is introduced into the process in an amount sufficient to produce the conversion of the organic sulphide. Generally speaking, this amount corresponds to a molar H₂S/thioether ratio of between 1 and 40, preferably between 2 and 30, more preferably between 2 and 10.

The reactants described above are contacted in the presence of a charge of the catalyst composition defined above in an appropriate reaction zone under reaction conditions appropriate for producing the desired thiol.

The process is preferably implemented in a reactor which is fed continuously with the reactants, although a batch reactor may also be used.

The reaction temperature varies according to the thioether used and the desired degree of conversion, but is generally situated within a range of between 50 and 350° C., preferably between 150 and 250° C.

The pressure at which the reaction is carried out also varies within wide limits. Commonly it is situated at between atmospheric pressure and 20 bars, preferably between 10 and 15 bars.

The contact time is generally between 1 and 50 s, preferably between 10 and 30 s.

The thioether employed in the process according to the invention may be the by-product obtained in a process for preparing thiol by adding hydrogen sulphide onto an alcohol or onto an olefin, in the presence of a catalyst and/or by photochemical activation. In this process version it is possible as a result advantageously to upgrade the said by-product.

The examples below are given purely by way of illustration of the invention, and must in no way be interpreted as constituting any limitation thereon. In these examples the abbreviation HPW corresponds to the 12-phosphotungstic acid of formula H₃PW₁₂O₄₀.nH₂O.

### EXAMPLE 1

### Preparation of the Pd catalyst and HPW, supported on SiO₂

For 200 g of SiO₂, an aqueous solution is prepared which contains 6 g of PdCl₂ and 140 g of HPW (weight expressed in equivalents of anhydrous acid, i.e. with n equal to 0).

The catalyst support used is an amorphous silica having a specific (or BET) surface area of 315 m²·g⁻¹, a pore diameter of the order of 12 to 14 nm and a pore volume of 1.6 cm³·g⁻¹. This support is treated under vacuum beforehand at a temperature of 100° C.

The solution obtained above is impregnated onto the support thus treated under vacuum by aspiration. When impregnation of the solution has been carried out, the mixture is stirred at atmospheric pressure for 1 hour.

The product obtained is dried under vacuum at ambient temperature and is then subjected to treatment with hydrogen at a temperature of 230° C. for the purpose of reducing the palladium.

The catalyst obtained is composed of 59% by weight of SiO₂, 1% by weight of Pd and 40% by weight of HPW.

### EXAMPLE 2

### Preparation of ethyl mercaptan (CH₃CH₂—SH) from diethyl sulphide (CH₃CH₂—S—CH₂CH₃):

A tubular reactor with a diameter of 25 mm is used which has a useful capacity of 200 ml and is charged with 200 ml of the catalyst composition prepared according to example 1.

Passed through this charge per hour are 120 g of diethyl sulphide (or 1 mol), 210 g of HₛS (or 5 mol) and 0.8 g of H₂ (or 0.08 mol).

The pressure in the reactor is maintained at 15 bars and the temperature is set at 235° C.

Continuous analysis of the crude reaction products shows that the initial conversion of the thioether is 52% , with an ethyl mercaptan yield of 49.3%.

### EXAMPLE 3

### Preparation of ethyl mercaptan (CH₃CH₂—SH) from diethyl sulphide (CH₃CH₂—S—CH₂CH₃)— change in the conversion of ethyl mercaptan over time:

Example 2 is repeated, continuing the sulfhydrolysis reaction for 6 days with the same charge of catalyst composition, and periodically (as a function of the time, expressed in days), measuring the conversion of diethyl sulphide (DES).

The results are collated in the table below.

TABLE 1

| Time (days) | Conversion of DES (in %) |
|---|---|
| 1 | 54 |
| 3 | 56 |
| 4 | 55 |
| 5 | 56 |
| 6 | 57 |

Table 1 shows that the catalyst system prepared in example 1 and used in the presence of hydrogen according to the process of the invention possesses good stability over time.

1. Process for preparing a mercaptan comprising contacting a thioether and hydrogen sulphide, in the presence of hydrogen and a catalyst composition comprising a strong acid and at least one metal selected from group VIII of the Periodic Table.

2. Process according to claim 1, wherein the strong, acid is selected from the group consisting of:
(a) one or mole heteropolyacids selected from the group H₃PW₁₂O₄₀.nH₂O, H₄SiW₁₂O₄₀.nH₂O or H₆P₂W₁₈O₆₂.nH₂O, in which n is an integer representing the number of molecules of water of crystallization, and is between 0 and 30, potassium, rubidium, caesium or ammonium salts thereof and mixtures of such salts;
(b) a sulphated zirconium oxide,
(c) a tulngstic zirconium oxide,
(d) a zeolite, and
(e) a cationic resin.

3. Process according to 1, wherein the strong acid is selected from the group potassium, rubidium, caesium or ammonium salts or a mixture of such salts of H₃PW₁₂O₄₀.nH₂O, H₄SiW₁₂O₄₀.nH₂O or H₆P₂W₁₈O₆₂.nH₂O, in which n is an integer representing the number of molecules of water of crystallization, and is between 0 and 30, a sulphated zirconium oxide, a tungstic zirconium oxide, a zeolite, and a cationic resin.

4. Process according to claim 1, wherein the catalyst composition comprises:
from 90% to 99.9%, by weight of strong acid, and
from 0.01% to 10%, by weight of at least one metal from group VIII.

5. Process according to claim 1, wherein the strong acid is a heteropolyacid selected from the group H₃PW₁₂O₄₀.nH₂O, H₄SiW₁₂O₄₀.nH₂O or H₆P₂W₁₈O₆₂.nH₂O, in which n is an integer representing the number of molecules of water of crystallization, and is between 0 and 30.

6. Process according to claim 5, wherein the catalyst composition comprises:
from 10% to 60%, by weight of strong acid,
from 0.01% to 10%, by weight of at least one metal from group VIII, and
from 30% to 80%, by weight of a support selected fibo silica SiO₂, alumina Al₂O₃, titanium dioxide TiO₂, zirconium oxide ZrO₂, and activated carbon.

7. Process according to claim 6, wherein the strong acid is 12-phosphotungstic acid.

8. Process according to one of to claim 1, wherein the at least one metal is selected from iron, cobalt, nickel, ruthenium, rhodium, palladium, osmium, iridium, and platinum.

9. Process according to claim 1, wherein the at least one metal is selected from palladium, ruthenium, and platinum.

10. Process according to claim 1, wherein the at least one metal is palladium.

11. Process according to claim 1 wherein the catalyst composition comprises approximately 40% by weight of 12-phosphotungstic acid, 1% of palladium and 59% of silica.

12. Process according to claim 1, wherein the hydrogen is introduced in an amount corresponding to a molar H₂S/H₂ ratio of between 10 and 200.

13. Process according to claim 1, wherein the thioether has the general formula:
R—S—R′ (I)
in which R and R′, which are identical or different, represent a linear or branched alkyl radical of 1 to 20 carbon atoms, or else a cycloalkyl radical of 3 to 7 carbon atoms.

14. Process according to claim 1, wherein the hydrogen sulphide is introduced in an amount corresponding to a molar H₂S/thioether ratio of between 1 and 40.

15. Process according to claim 1, wherein the catalyst composition comprises:
from 98.5% to 99.9%, by weight of strong acid, and
from 0.05% to 1.5%, by weight of at least one metal from group VIII.

16. Process according to claim 5, wherein the catalyst composition comprises:
from 25 to 50%, by weight of strong acid,
from 0.1% to 2%, by weight of at least one metal from group VIII, and
from 48% to 75%, by weight of a support selected from silica SiO₂, alumina Al₂O₃, titanium dioxide TiO₂, zirconium oxide ZrO₂, and activated carbon.

17. Process according to claim 1, wherein the hydrogen is introduced in an amount corresponding to a molar H₂S/H₂ ratio of between 50 and 100.

18. Process according to claim 1, wherein the hydrogen sulphide is introduced in an amount corresponding to a molar H₂S/thioether ratio of between 2 and 30.

19. Process according to claim 1, wherein the hydrogen sulphide is introduced in an amount corresponding to a molar H₂S/thioether ratio of between 2 and 10.

20. Process according to claim 1, wherein n is between 6 and 20.

21. Process according to claim 7, wherein said 12-phosphotungstic acid is impregnated on silica.

22. Process according to claim 13, wherein said linear or branched alkyl radical has 1 to 12 carbon atoms.
